(19) United States
(12) Patent Application Publication
(10) Pub. No.: US 2005/0090712 A1
(43) Pub. Date: Apr. 28, 2005

(54) Res-Q-Scope

(76) Inventor: Anthony Cubb, Kingwood, TX (US)

Correspondence Address: Anthony Cubb, M.D., 19714 Texas Laurel Trail, Kingwood, TX, 77346, US

(21) Appl. No.: 10/693,206

(22) Filed: Oct. 23, 2003

(51) Int. Cl. A61B 1/00

(52) U.S. Cl. 600/120; 600/153

(57) ABSTRACT

A multiple function laryngoscope to be used for the safe intubation of a patient's trachea during a respiratory emergency or as an elective procedure. A two section instrument. Proximally, a reusable handle that houses a rechargeable battery, electronic circuits that feed a distal digital image to variable position LCD screen or viewing port, switches and low battery indicator light. Distally, the handle electrically couples with a disposable curved scabbard. The scabbard features a dorsal endotracheal tube channel with a wavy opening which allows preloading and gentle extraction of different size endotracheal tubes once the patient's trachea has been intubated. The scabbard's distal end strategically houses a distal sweeper that engages the epiglottis and exposes the glottis, an opening for the exit of a preloaded endotracheal tube, a LED light, a suction/oxygenation port, and a lens coupled with a digital imaging system (CMOS) to digitize and transport a distant wide field of view. Safe LCD view of one or serial rapid intubations are possible by rapidly replacing for a clean disposable scabbard in case of multiple emergencies.

### FIELD OF THE INVENTION

The present invention generally relates to an instrument for accessing the laryngeal area of the human body and, more particularly, to an improved laryngoscope to use in the respiratory emergency field and hospital setting for safe, indirect, visual endotracheal intubation.

### BACKGROUND OF THE INVENTION

Laryngoscopes are widely known and used in the medical field to facilitate endotracheal intubation of a patient during a respiratory emergency situation in order to provide airway patency and positive air pressure through the upper airway and the lungs, manually or through mechanical ventilation of the lungs to the injured person. Such laryngoscopes are also used during surgical procedures to maintain an open airway and provide ventilatory support during surgery under anesthesia. In the human anatomy, the epiglottis normally overlies the glottis opening into the larynx to prevent the passage of food into the trachea during eating. Thus, when undertaking an endotracheal intubation, it is necessary to displace the epiglottis from the glottal opening to permit the air tube to be inserted in between the vocal cords and subsequently into the trachea.

Various laryngoscope constructions are known. The more widely used laryngoscopes consist of an elongate, rigid metal blade which is supportably attached to a handle. These types of laryngoscopes are inserted through the mouth of the patient into the pharyngeal area to displace the tongue and epiglottis forcibly in the upwards direction and most often permitting direct visualization of the glottis through the mouth opening. Since the mouth and laryngeal area are at an approximate 90 degrees to each other, such laryngoscopes require that the patient's head be hyper extended in the backwards direction to create a relatively straight path to permit direct visualization of the glottis by the operator of the laryngoscope. Substantial force by the operator is required to overcome the natural skeleto-musculature tendencies of the patient and the operator is required to perform the procedure while located at the head of the patient. Additionally, if there is concern that the patient may have suffered spinal injuries, the technique for possible direct visualization of the glottis involves risk due to the potential for increased spinal injury from this procedure.

Without visualization of anatomical structure, intubation of a patient during an emergency situation may require blind placement of an endotracheal tube based on free hand trial and error. Without proper positioning and guidance, the tubular members often cause trauma or injury to anatomical tissue, or missed intubation into the esophagus with potential fatal consequences.

Surgical instruments having means for indirect illumination and visualization of the pharyngeal areas of the body are known. U.S. Pat. Nos. 3,776,222 and 3,913,568 disclose devices for endotracheal intubation which comprise flexible or articulatable tubular probes having internal fiber optics for lighting and viewing the internal areas of the body. As disclosed in those patents, the probes carry a slidably removable endotracheal tube surrounding their outer surfaces and the probe is directly inserted into the trachea to position the tube. Such devices obviously require the use of relatively large diameter endotracheal tubes in order to be carried on the tubular probe, and their use necessarily is limited to patients with sufficiently large airway passages to accommodate the combined size of the probe and endotracheal tube. Additionally, due to the flexible nature of the probes, it is difficult to manipulate the probe to displace the tongue and epiglottis to permit guidance during insertion of the tube into the trachea. These instruments require a high degree of skill and a concomitant degree of training to perform the procedure quickly, without injuring the patient. Additionally, because of expense, lack of portability, and sterilization requirements to prevent cross contamination among patients, these instruments are generally not available in a non-hospital setting.

As a consequence, there has been a long felt need for a device which can facilitate intubation so as to quickly and accurately accomplish indirect visual endotracheal intubation by manipulation of soft tissue in the mouth without needing to overcome the natural skeleto-musculature tendencies of the patient. There is a further need to for such a device which provides indirect visualization of the surrounding anatomical structures from a diversity of orientations relative to the patient being intubated. There is a further need also for such a device which is disposable and inexpensive enough to be financially accessible for any emergency vehicle or field use, and which is easily and efficiently used by a practitioner with basic training, particularly for patents having a short, obese neck with a commonly anteriorly located upper airway. The need exists for an intubating device which provides clear and external imagery of upper laryngeal structures for ease of viewing by the practitioner. There is a further need for such a device which can effectively accommodate different diameters of intubation tubes, according to the individual patient's needs. There is a further need for such a device which can transport a pre-loaded endotracheal tube and place it at a ready position for final insertion from a short distance from the target organs, the vocal cords. Lastly, there is a further need for an intubating device which is partially or fully disposable for minimizing the potential cross contamination between patients due to poor sterilization or poor cleaning of conventional equipment during respiratory emergencies involving multiple victims with severe respiratory compromise necessitating multiple, simultaneous and/or rapidly sequential intubations.

### SUMMARY OF THE INVENTION

The present invention is designed to overcome the aforementioned difficulties during intubation by providing a disposable, inexpensive, easily used and efficient endotracheal intubation device, designed for ready manipulation of oral cavity soft tissue during placement to minimize the effort of both patient and operator, containing a bright light source and indirect visualization system composed of either fiberoptic bundles, a digital imaging system or a combination thereof, simultaneous suctioning ability, and external viewing mechanism which can be placed at a multiplicity of positions relative to the patient or allow for a multiplicity of locations of the operator relative to the patient that enables an operator to visualize the anatomical structure in front of the end point of the intubation device insertion from any orientation of the practitioner relative to the patient during insertion of the device and/or endotracheal tube of varying size.

In one preferred embodiment, an endotracheal intubation device is provided including an optical assembly enclosed by a housing anatomically accommodating to the operator's hand where the optical assembly includes an image conducting system having a curved distal portion and extending from a first end of the housing, and a proximal portion and extending from a second end of the housing through an image viewing mechanism pivotally attached at a first end of the viewing mechanism to the second end of the housing. The image viewing mechanism has at its second end a viewing system wherein the image conducting system optically communicates with the viewing system through the image viewing mechanism. The ball joint of the image viewing mechanism can be manipulated by application of light force applied to the image viewing mechanism such that a plurality of angular orientations with respect to the housing are achieved. Positioned at the second end of the image viewing mechanism is a viewing system which optically communicates with the image conducting system. The viewing system provides for accurate viewing by the practitioner when the practitioner's eye is within a comfortable field of vision of the operator from the viewing port.

A scabbard is attached to the housing which is sized to sealably receive a portion of the first end of the housing and the distal end of the image conducting system. The scabbard comprises a curved structure having a terminal edge surface and a plurality of spaced conduits extending through the scabbard. The terminal edge exhibits a short prolongation of the bottom side of the structure for manipulating and holding the epiglottis away from covering the vocal cords to clear and expose the target anatomical structures for intubation. A first one of the conduits extends longitudinally through the scabbard and houses the image conducting system with the distal end of the conduit optically open end at the terminal edge surface of the scabbard, with the terminal surface of the conduit opening sealed by a final lens. A second one of the conduits extends along an outer surface portion of the scabbard and defines an open serpentginous channel that is sized to removably receive multiple sizes of an endotracheal tube, ready for accurate disposition in the patient. A third one of the conduits extends longitudinally through the scabbard and provides a suction path for the operator when attached to a vacuum source.

A power source is electrically connected to the image conducting system to provide an illuminated area at the terminal edge surface of the scabbard and for transmission of images from the illuminated area to the viewing port located at said proximal end of the image conducting system.

### BRIEF DESCRIPTION OF THE DRAWINGS

These and other features and advantages of the present invention will be more fully disclosed in, or rendered obvious by, the following detailed description of the preferred embodiment of the invention, which is to be considered together with the accompanying drawings wherein like numbers refer to like parts further wherein:

FIG. 1 is a side view of an endotracheal intubation device having a multi-positional viewing mechanism formed in accordance with the present invention;

FIG. 2 is a top view of the endotracheal intubation device shown in FIG. 1;

FIG. 3 is a longitudinal cross-sectional view of the endotracheal intubation device shown in FIG. 1, showing an endotracheal tube, an image conducting system, and a suction tube positioned with their respective conduits;

FIG. 4 is a housing body cross-sectional view of the endotracheal intubation device shown in FIG. 1, showing the cross-section of an electrical power source for the image conducting system;

FIG. 5 is a cross-section of the scabbard of the endotracheal intubation device of FIG. 1; and

FIG. 6 is an insertion end view of the scabbard of the endotracheal intubation device of FIG. 1.

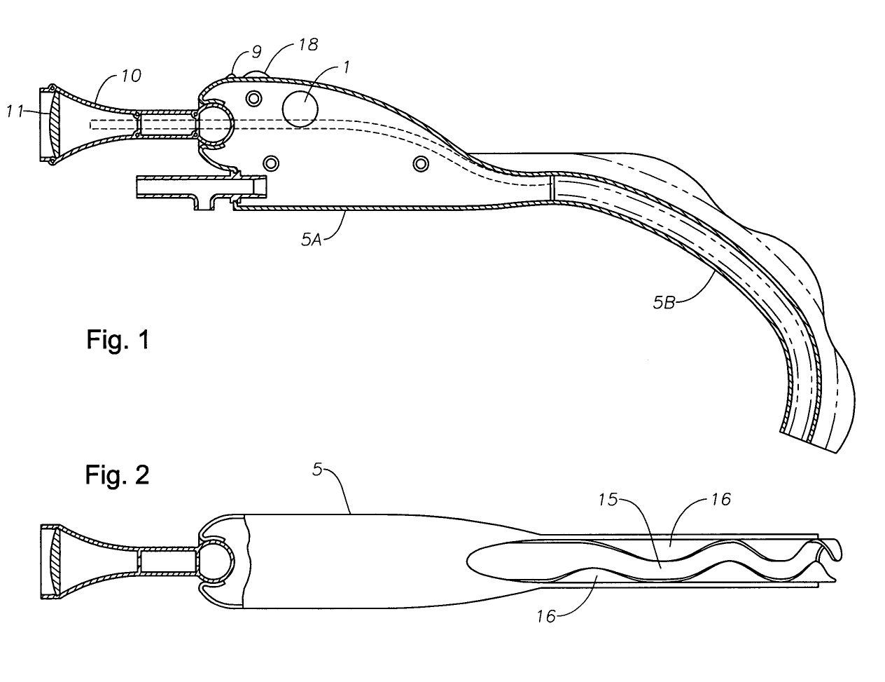

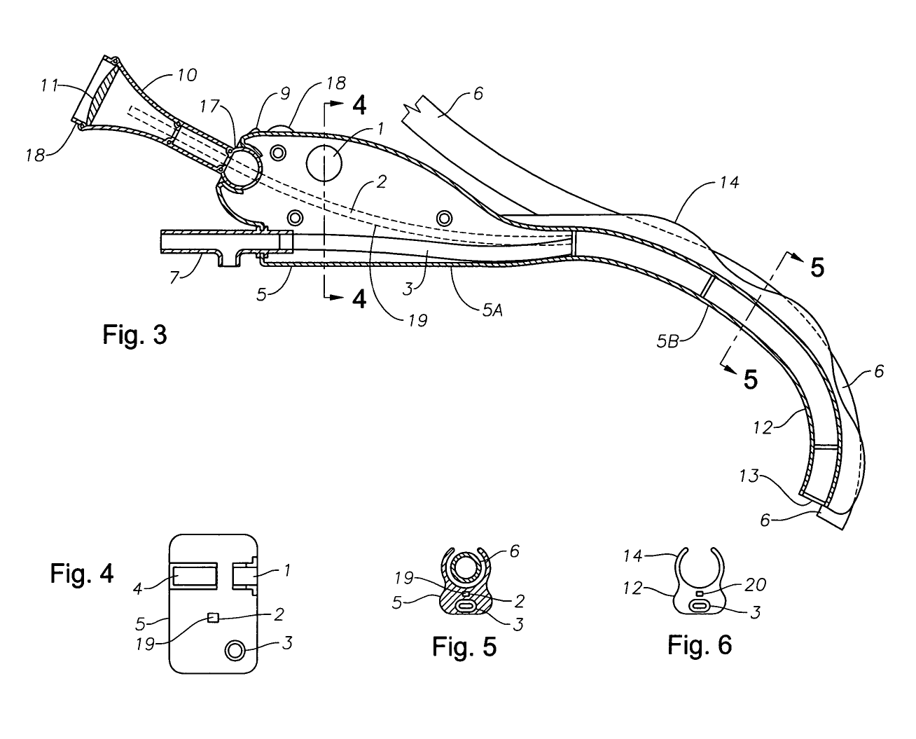

### DETAILED DESCRIPTION OF PREFERRED EMBODIMENT

This description of preferred embodiments is intended to be read in connection with the accompanying drawings, which are to be considered part of the entire written description of this invention. The drawing figures are not necessarily to scale and certain features of the invention may be exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. In the claims, means-plus-function clauses are intended to cover the structures described, suggested or rendered obvious by the written description or drawings for performing the recited function, including not only structural equivalents but also equivalent structures.

The present invention provides an endotracheal intubation device or intubator 5 that includes a pivotal viewing mechanism 10 which enables indirect visualization of a patient's upper airway from multiple operator positions relative to the patient orientation. The intubator 5 includes a scabbard 5B and an optical assembly enclosed by a housing anatomically accommodating to the operator's hand 5A. Scabbard 5B is formed from a hard polymer material formed so as to generally comprise the curved shape of the anatomical contour of the tongue and a similar curvature seen in a conventional intubation blade, e.g., a Macintosh or Miller (straight) blade. Scabbard 5B includes a curved distal end 12. An image conducting passageway 2 extends throughout the length of intubator 5, and opens at the terminal face 13 (FIG. 6) of curved distal end 12 of scabbard 5B. An image conducting system is housed in image conducting passageway 2, and communicates with viewing system 11 of positional viewing mechanism 10. A second passageway 3 extends throughout the length of scabbard 5B in substantially parallel relation to image conducting passageway 2, and also opens at terminal face 13 of curved distal end 12. A port 7 is arranged on the lower side of optical housing assembly 5A, in fluid flow communication with second passageway 3. Direct suction may be applied to port 7 so that foreign material and secretions from the throat may be transferred through second passageway 3 thereby eliminating the need for suction catheters or the like. A channel formed on the outer curved surface of distal end 12 of scabbard 5B provides an endotracheal tube receptacle 14. Tube receptacle 14 is sized so as to snugly, but releasably accommodate an endotracheal tube 6 of the type, and range of sizes well known in the art. Such a tube 6 may be prepositioned within tube receptacle 14 ready for endotracheal intubation. A top opening 15 is serpentginous as defined by a plurality of spaced-apart, interdigitated fingers 16, which aid in snugly but releasably maintaining tube 6 within tube receptacle 14 during insertion of scabbard 5B into a patient's mouth and throat.

Optical housing assembly 5A includes a positional viewing mechanism 10 connected by means of a pivot mechanism 17 and a viewing system 11 which is optically connected to the image conducting system 19 contained in image conducting passageway 2. Optical housing assembly 5A also includes a battery power supply source 4 electrically connected to the image conducting system 2 and to a charge indicator 9. Battery power supply source 4 is electrically connected to an off/on button 18 arranged so as to be easily accessible to an operator. Image conducting system 19 comprises a fiber optic bundle, a digital conducting system or a combination of both. Image conducting system 19 is interconnected to the light source (not shown) within optical housing assembly 5A, while the distal end terminates at lens 20 or similar light conditioning or focusing device being sealingly disposed over the open end of optical passageway 2 at terminal face 13 of curved distal end 12 of scabbard 5B, or over the free end of image conducting system 19. This arrangement has the added benefit of preventing bodily fluids and the like from entering optical passageway 2 and contaminating image conducting system 19. Image conducting system 19 extends from the light source within optical housing assembly 5A, and is optically interconnected with positional viewing mechanism 10. Positional viewing mechanism 10 comprises an viewing port 18 disposed at one end by means of pivot mechanism 17, and provides for ease of visualization of the larynx and surrounding structures by the operator.

Viewing port 18 supports a viewing system 11 which provides for accurate viewing by the practitioner within a comfortable distance between the practitioner's eye and the viewing system 11. Image conducting system 19 extends from the light source within optical housing assembly 5A, and to viewing port 18 through positional viewing mechanism 10, and optically communicates with viewing system 11. Positional viewing mechanism 10 pivotally attaches to optical housing assembly 5A by means of a pivot mechanism 17 with a sufficiently frictional fit for supporting and maintaining positional viewing mechanism 10 in a desired position and orientation so as to allow for indirect visualization of a patient's airway from a wide range of positions relative to the orientation of the patient's head.

An intubation tube 6 is positioned within tube receptacle 14 of the scabbard 5B by sliding tube 6 downwardly through the serpentginous open channel 15 formed by the interdigitated fingers 16 so that it is held releasably in place within the outer, dorsal portion of scabbard 5B. Once this assembly is completed, an intubation procedure may be begun.

More particularly, and unlike conventional intubation devices, the patient's head need not be manipulated with a face-chin lifting maneuver. Only the lower jaw needs to be somewhat distended, and the mouth open sufficiently to introduce endotracheal intubation device 5. Curved distal end 12 of scabbard 5B is then inserted through the mouth into the throat passageway, so as to displace the soft tissue of the tongue and epiglottis, and expose the glottis of the patient. Once in this position, suction may be applied to port 7, so as to draw bodily secretions and fluids away from the glottis and larynx through second passageway 3. Advantageously, this procedure may be visualized via positional viewing mechanism 10 by pivoting so as to position viewing port housing 18 at a location convenient for the person performing the intubation to clearly observe the intubation at a safe distance from the patients mouth and within a comfortable distance between the practitioner's eye and the viewing system 11. It will be understood that positional viewing mechanism 10 may be maneuvered into a plurality of positions, as needed, to provide for the safe and comfortable access by the person performing the intubation and to allow for viewing of the anatomical structures and devices so as to provide control in the intubation process.

With the patient's larynx in view through viewing port 18, tube 6 is maneuvered through the larynx and into the trachea of the patient, all the while being observed by the person performing the intubation.

Once tube 6 has been properly positioned within the trachea, the endotracheal intubation device 5 is removed from the patient's mouth by the operator, while holding tube 6 in place, and sliding scabbard 5B along tube 6 until the scabbard 5B exits the patient's mouth and tube 6 can be removed from the tube receptacle 14.

Advantages of the Invention

Numerous advantages are obtained by employing the present invention.

More specifically, an endotracheal intubation device is provided which avoids many of the aforementioned problems associated with prior art devices.

In addition, an endotracheal intubation device is provided which allows the operator to be positioned not only at the patient's head while performing an intubation (which is the conventional preferred position with very limited options), but also permits endotracheal tube placement under indirect visualization of the target area, the larynx, from different positions relative to the patient's location and orientation.

Furthermore, an endotracheal intubation device is provided in which an emergency care provider may no longer need to reposition or manipulate the neck to facilitate visualization of the vocal cords, such as in the case of an automobile accident when the person is in need of respiratory assistance and there is a potential threat of neck or cervical spine injuries, which can lead to further neck, spine, and spinal cord damage, and even paralysis by repositioning of the patient's neck.

Also, an endotracheal intubation device is provided which does not require the manipulation of the neck, injured or not, to visualize the vocal cords and other anatomical structures, thus alleviating previous intubation effort related problems, such as broken or chipped teeth.

In addition, an endotracheal intubation device is provided, including a viewing port which allows the visualization of the larynx and associated structures, outside the patient's mouth and readily accessible to the operator's field of view. Further, the viewing port can be oriented at multiple positions about the patient's mouth and head, so as to allow the operator to intubate in tight or narrow spaces often seen in accident scenes when conventional emergency care is presently unable to do so with portable equipment. This is extremely helpful when a victim is trapped inside of a vehicle where they would normally not be able to be intubated.

Furthermore, an endotracheal intubation device is provided that permits intubation from the side of a bed in a medical care facility. Previously, such intubation had to be performed from only the head of the bed, necessitating removal of the headboard of the bed to create a space between the bed and the wall, and the person maneuvering him/herself through the numerous intravenous lines and monitor equipment to be able to stand between the wall and the head of the bed, in order to place the endotracheal tube within the patient's trachea.

In addition, an endotracheal intubation device is provided which is designed to provide disposability of the parts exposed to the patient's tissue and bodily fluids, thus minimizing or eliminating the possibility of cross contamination between patients or the possibility of exposure of one patient by another patient infected by diseases such as HIV, hepatitis, tuberculosis, among others.

In addition, an endotracheal intubation device is provided which is portable and easily used in emergency medical situations involving multiple victims.

In addition, an endotracheal intubation device is provided which is designed for visualization of the patient's anatomical structures of the throat and upper airway to minimize missed intubations, particularly those into the esophagus with possible fatal consequences.

In addition, an endotracheal intubation device is provided which permits intubation from a variety of positions of the operator relative to the patient, overcoming a common problem in accident and other emergency situations.

In addition, an endotracheal intubation device is provided which is anatomically contoured to avoid local trauma to the patient during insertion.

In addition, an endotracheal intubation device is provided which allows the operator to perform the insertion procedure at a distance from the patient which lessens the operator's exposure to the spread of infectious diseases and other bacterial matter by not having to hyper extend the neck to see the vocal cords, and thereby decreasing the risk of the patient's coughing up bodily secretions into the operator's eyes and face.

In addition, an endotracheal intubation device is provided which allows for the simultaneous suctioning of bodily fluids from the throat area to eliminate the obstruction from the field of view necessary for intubation or to reversibly provide oxygen to the patient from a pressure source.

In addition, an endotracheal intubation device is provided which requires minimal physical force on the part of the operator to expose the upper airway and larynx for expedient intubation.

In addition, an endotracheal intubation device is provided which provides an enhanced visual imaging system to assure ease of observation of anatomical structures at viewing port.

It is to be understood that the present invention is by no means limited only to the particular constraints herein disclosed and shown in the drawings, but also comprises any modifications or equivalents within the scope of the claims.

1. An endotracheal intubation device comprising:
an optical housing assembly including an image conducting system having a distal portion and extending from a first end of said optical housing assembly, and a proximal portion and extending from a second end of said optical housing assembly through a positional viewing mechanism pivotally attached at a first end of said positional viewing mechanism to said second end of said optical housing assembly. Said positional viewing mechanism having at its second end a viewing system wherein said image conducting system optically communicates with said viewing system through said positional viewing mechanism;
a scabbard sized to sealably receive a portion of said first end of said optical housing assembly and said image conducting system, wherein said scabbard comprises a curved structure having a terminal edge surface and a plurality of spaced conduits extending through said scabbard wherein: (i) a first one of said conduits extends longitudinally through said scabbard and has at least one optically open end at said terminal edge surface of said scabbard, (ii) a second one of said conduits extends along an outer surface portion of said scabbard and defines an open channel that is sized to removably receive an endotracheal tube and comprises two open ends, one of which opens onto said terminal edge surface of said scabbard to provide a predictable point and direction for insertion of said endotracheal tube, and (iii) a third one of said conduits extends longitudinally through said scabbard and comprises two open ends, a first end of which opens onto said terminal edge surface of said scabbard and a second end of which being adapted to for connection to a vacuum or supplemental oxygen providing source; and
a power source electrically connected to said image conducting system to provide an illuminated area at said terminal edge surface of said scabbard from said distal portion of said image conducting system and for transmission of images from the said illuminated area to said positional viewing mechanism located at said proximal end of said image conducting system.

2. An endotracheal intubation device according to claim 1 wherein said scabbard is formed from a polymer so as to comprise an intubation blade like structure for inserting into a patient's mouth, formed so as to generally comprise the shape of the anatomical contour of a patient's tongue.

3. An endotracheal intubation device according to claim 1 having a lens sealingly disposed over said open end of said first one of said conduits on said terminal edge surface, and illuminatingly in communication with said distal end of said image conducting system.

4. An endotracheal intubation device according to claim 1 wherein said power source electrically connected to said image conducting system is a battery.

5. An endotracheal intubation device according to claim 1 wherein said open channel provides a serpentginous open channel that is sized so as to firmly, but releasably accommodate a plurality of sizes of said endotracheal tube.

6. An endotracheal intubation device according to claim 1 wherein said open channel is partially obstructed by a plurality of spaced-apart, interdigitated fingers so as to firmly, but releasably accommodate a plurality of sizes of said endotracheal tube.

7. An endotracheal intubation device according to claim 1 wherein said image conducting system is elongate and flexible.

8. An endotracheal intubation device according to claim 1 wherein said image conducting system is an optical fiber bundle.

9. An endotracheal intubation device according to claim 1 wherein said image conducting system is a digital conducting system.

10. An endotracheal intubation device according to claim 1 wherein said image conducting system is a combination of an optical fiber bundle and a digital conducting system.

11. An endotracheal intubation device according to claim 1 wherein said positional viewing mechanism's said positional attachment is produced with a sufficiently frictional fit for supporting and maintaining said positional viewing mechanism in a desired position and orientation.

12. An endotracheal intubation device according to claim 1 comprising a port arranged at said first end of said scabbard in fluid flow communication with said second conduit.

13. An endotracheal intubation device according to claim 1 wherein said optical housing assembly has an exterior surface with curvature to effectively accommodate the hand grip of an operator.

14. An endotracheal intubation device according to claim 1 wherein said positional viewing mechanism allows for accurate visualization of the patient's target organs by the operator at a distance within a comfortable field of vision of the operator from said positional viewing mechanism.

15. An endotracheal intubation device according to claim 1 wherein said scabbard is detachable from said first end of said optical housing assembly and is replaceable or disposable.
